(19) United States
(12) Patent Application Publication
(10) Pub. No.: US 2007/0073151 A1
(43) Pub. Date: Mar. 29, 2007

(54) Automated imaging and therapy system

(75) Inventor: Warren Lee, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/225,331

(22) Filed: Sep. 13, 2005

(51) Int. Cl. A61B 8/00

(52) U.S. Cl. 600/439

(57) ABSTRACT

A system for imaging and providing therapy to one or more regions of interest is presented. The system includes an imaging and therapy catheter configured to image an anatomical region to facilitate assessing need for therapy in one or more regions within the anatomical region and delivering therapy to the one or more regions of interest within the anatomical region. In addition, the system includes a medical imaging system operationally coupled to the catheter and having a display area and a user interface area, wherein the medical imaging system is configured to facilitate defining a therapy pathway to facilitate delivering therapy to the one or more regions of interest.

### BACKGROUND

The invention relates generally to diagnostic imaging, and more particularly to automated imaging and ablation therapy.

Heart rhythm problems or cardiac arrhythmias are a major cause of mortality and morbidity. Atrial fibrillation is one of the most common sustained cardiac arrhythmia encountered in clinical practice. Cardiac electrophysiology has evolved into a clinical tool to diagnose and treat these cardiac arrhythmias. As will be appreciated, during electrophysiological studies, multipolar catheters are positioned inside the anatomy, such as the heart, and electrical recordings are made from the different chambers of the heart. Further, catheter-based ablation therapies have been employed for the treatment of atrial fibrillation.

Conventional techniques utilize radio frequency (RF) catheter ablation for the treatment of atrial fibrillation. Currently, catheter placement within the anatomy is typically performed under fluoroscopic guidance. Intracardiac echocardiography has also been employed during RF catheter ablation procedures. Additionally, the ablation procedure may necessitate the use of a multitude of devices, such as a catheter to form an electroanatomical map of the anatomy, such as the heart, a catheter to deliver the RF ablation, a catheter to monitor the electrical activity of the heart, and an imaging catheter. A drawback of these techniques however is that these procedures are extremely tedious requiring considerable manpower, time and expense. Further, the long procedure times associated with the currently available catheter-based ablation techniques increase the risks associated with long term exposure to ionizing radiation to the patient as well as medical personnel.

Additionally, with RF ablation the tip of the catheter is disadvantageously required to be in direct contact with each of the regions of the anatomy to be ablated. RF energy is then used to cauterize the identified ablation sites. Further, in RF ablation techniques, the catheter is typically placed under fluoroscopic guidance, However, fluoroscopic techniques disadvantageously suffer from drawbacks, such as difficulty in visualizing soft tissues, which may result in a less precise definition of a therapy pathway. Consequently, these RF ablation techniques typically result in greater collateral damage to tissue surrounding the ablation sites. In addition, RF ablation is associated with stenosis of the pulmonary vein.

Moreover, a pre-case computed tomography (CT) and/or magnetic resonance imaging (MRI) as well as electroanatomical (EA) mapping systems may be employed to acquire static, anatomical information that may be used to guide the ablation procedure. However, these systems disadvantageously provide only static images and are inherently unfavorable for imaging dynamic structures such as the heart.

There is therefore a need for an integrated system for performing ablation procedures. In particular, there is a significant need for a design that advantageously integrates the imaging, ablation and mapping aspects of the ablation procedure thereby eliminating the need for harmful exposure to fluoroscopy and pre-case CT/MRI and static EA mapping systems. Additionally, there is a particular need for optimizing ablation pathway guidance and visualization of anatomy being imaged.

### BRIEF DESCRIPTION

Briefly, in accordance with aspects of the present technique, a system for imaging and providing therapy to one or more regions of interest is presented. The system includes an imaging and therapy catheter configured to image an anatomical region to facilitate assessing need for therapy in the one or more regions of interest within the anatomical region and delivering therapy to the one or more regions of interest within the anatomical region. In addition, the system includes a medical imaging system operationally coupled to the catheter and having a display area and a user interface area, wherein the medical imaging system is configured to facilitate defining a therapy pathway to facilitate delivering therapy to the one or more regions of interest.

In accordance with another aspect of the present technique a method for imaging and providing therapy to one or more regions of interest is presented. The method includes generating an image from acquired image data for display on a display area of a medical imaging system. Further, the method includes identifying one or more regions of interest requiring therapy on the displayed image. The method also includes defining a therapy pathway in response to the identified one or more regions of interest. Additionally, the method includes delivering therapy to the one or more regions of interest in accordance with the defined therapy pathway. Computer-readable medium that afford functionality of the type defined by this method is also contemplated in conjunction with the present technique.

In accordance with further aspects of the present technique a system for imaging and providing therapy to one or more regions of interest is presented. The system includes an imaging and therapy catheter configured to image an anatomical region to facilitate assessing need for therapy in the one or more regions of interest within the anatomical region and delivering therapy to the one or more regions of interest within the anatomical region. In addition, the system includes a medical imaging system operationally coupled to the imaging and therapy catheter and having a display area and a user interface area, wherein the medical imaging system is configured to facilitate defining a therapy pathway to facilitate delivering therapy to the one or more regions of interest. The system also includes an image generation sub-system for receiving acquired image data, generating an image of the anatomical region and displaying the image on the display area of the medical imaging system. Further, the system includes an operator console for identifying the one or more regions of interest on the displayed image.

### DRAWINGS

These and other features, aspects, and advantages of the present invention will become better understood when the following detailed description is read with reference to the accompanying drawings in which like characters represent like parts throughout the drawings, wherein:

FIG. 1 is a block diagram of an exemplary ultrasound imaging and therapy system, in accordance with aspects of the present technique;

FIG. 2 is a front view of a display area of the imaging and therapy system of FIG. 1, in accordance with aspects of the present technique;

FIG. 3 is an illustration of an exemplary imaging and therapy transducer for use in the system illustrated in FIG. 1, in accordance with aspects of the present technique;

FIG. 4 is an illustration of another exemplary imaging and therapy transducer for use in the system illustrated in FIG. 1, in accordance with aspects of the present technique; and

FIG. 5 is a flow chart illustrating an exemplary process of imaging and providing therapy to one or more regions of interest, in accordance with aspects of the present technique.

### DETAILED DESCRIPTION

As will be described in detail hereinafter, an automated image-guided therapy system and method in accordance with exemplary aspects of the present technique are presented. Based on image data acquired by the image-guided therapy system via an imaging and therapy catheter, a user may assess need for therapy in an anatomical region and use a human interface device, such as a mouse, to direct the therapy via the image-guided therapy system.

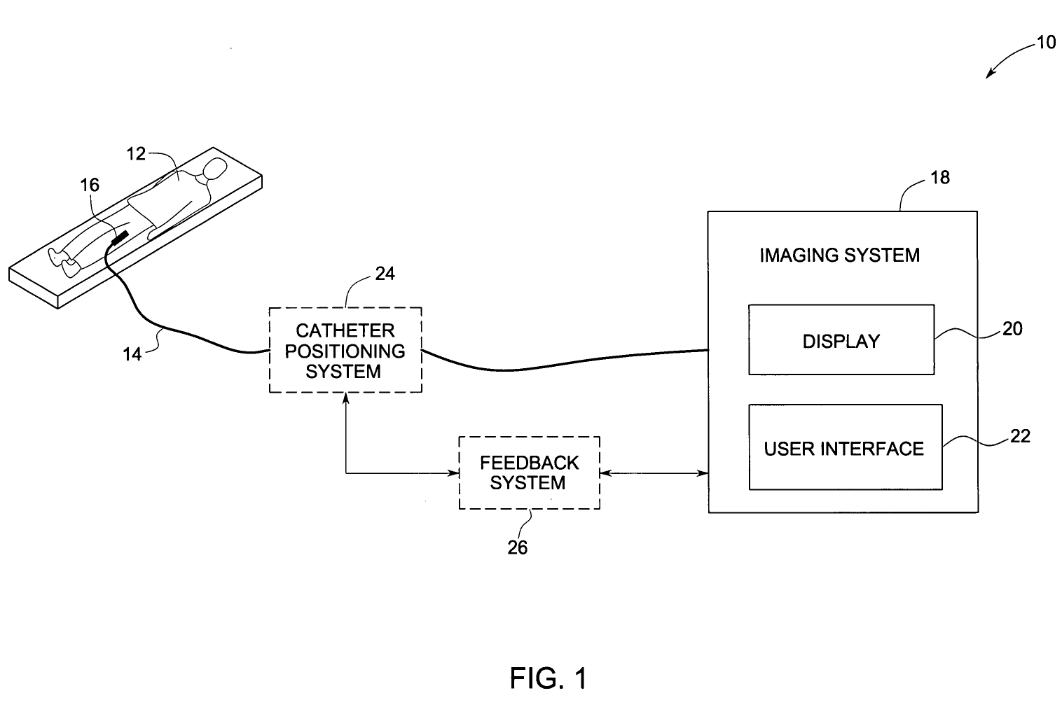

FIG. 1 is a block diagram of an exemplary system 10 for use in imaging and providing therapy to one or more regions of interest in accordance with aspects of the present technique. The system 10 may be configured to acquire image data from a patient 12 via an imaging and therapy catheter 14. As used herein, “catheter” is broadly used to include conventional catheters, transducers or devices adapted for applying therapy. Further, as used herein, “imaging” is broadly used to include two-dimensional imaging, three-dimensional imaging, or preferably, real-time three-dimensional imaging. Reference numeral 16 is representative of a portion of the imaging and therapy catheter 14 disposed inside the vasculature of the patient 12.

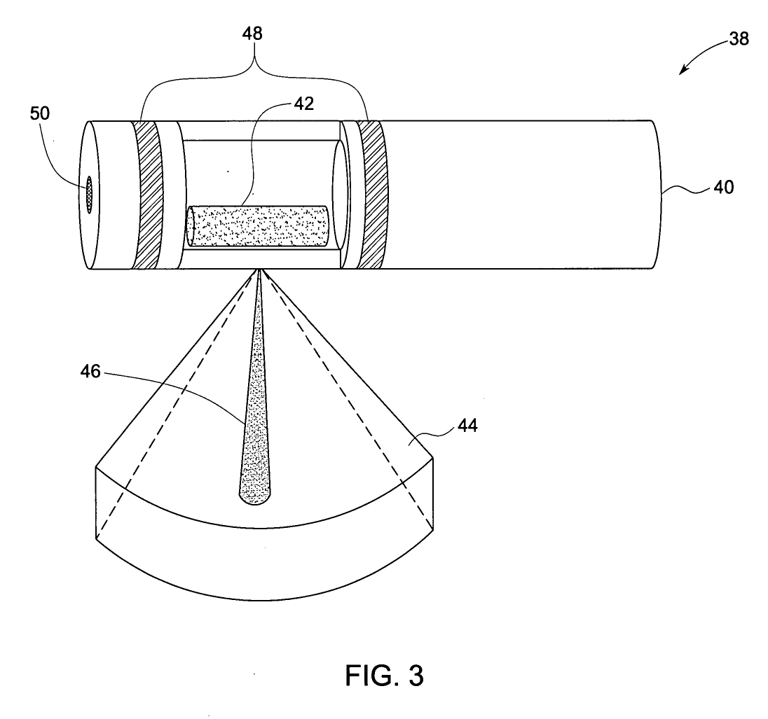

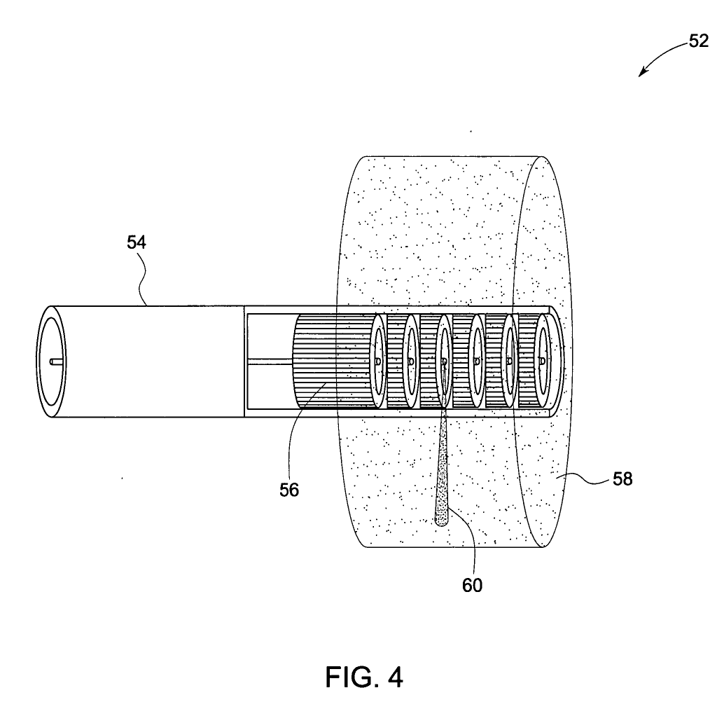

In certain embodiments, an imaging orientation of the imaging and therapy catheter 14 may include a forward viewing catheter or a side viewing catheter. However, a combination of forward viewing and side viewing catheters may also be employed as the imaging and therapy catheter 14. The imaging and therapy catheter 14 may include a real-time imaging and therapy transducer (not shown). According to aspects of the present technique, the imaging and therapy transducer may include integrated imaging and therapy components. Alternatively, the imaging and therapy transducer may include separate imaging and therapy components. The imaging and therapy transducer will be described in greater detail with reference to FIGS. 3-4. It should be noted that although the embodiments illustrated are described in the context of a catheter-based transducer, other types of transducers such as transesophageal transducers or transthoracic transducers are also contemplated.

In accordance with aspects of the present technique, the imaging and therapy catheter 14 may be configured to image an anatomical region to facilitate assessing need for therapy in one or more regions of interest within the anatomical region of the patient 12 being imaged. Additionally, the imaging and therapy catheter 14 may also be configured to deliver therapy to the identified one or more regions of interest. As used herein, “therapy” is representative of ablation, percutaneous ethanol injection (PEI), cryotherapy, and laser-induced thermotherapy. Additionally, “therapy” may also include delivery of tools, such as needles for delivering gene therapy, for example. Additionally, as used herein, “delivering” may include various means of providing therapy to the one or more regions of interest, such as conveying therapy to the one or more regions of interest or directing therapy towards the one or more regions of interest. As will be appreciated, in certain embodiments the delivery of therapy, such as RF ablation, may necessitate physical contact with the one or more regions of interest requiring therapy. However, in certain other embodiments, the delivery of therapy, such as high intensity focused ultrasound (HIFU) energy, may not require physical contact with the one or more regions of interest requiring therapy.

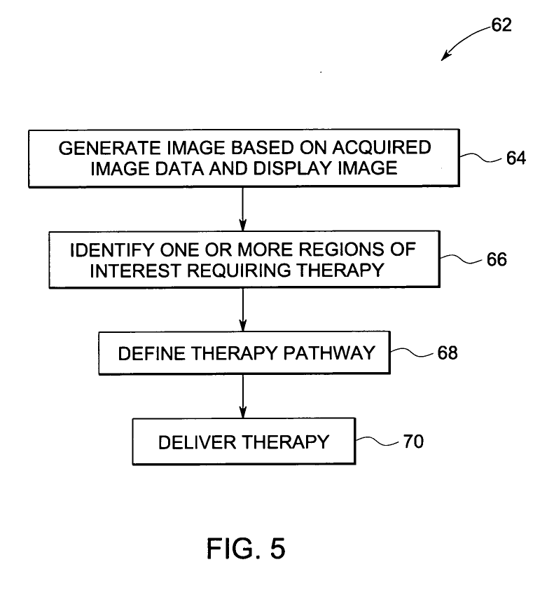

The system 10 may also include a medical imaging system 18 that is in operative association with the imaging and therapy catheter 14 and configured to define a therapy pathway to facilitate delivering therapy to the one or more regions of interest. The imaging system 10 may be configured to define the therapy pathway in response to user input or automatically define the therapy pathway as will be described in greater detail with reference to FIG. 5. Accordingly, in one embodiment, the medical imaging system 18 may be configured to provide control signals to the imaging and therapy catheter 14 to excite the therapy component of the imaging and therapy transducer and deliver therapy to the one or more regions of interest. In addition, the medical imaging system 18 may be configured to acquire image data representative of the anatomical region of the patient 12 via the imaging and therapy catheter 14.

As illustrated in FIG.1, the imaging system 18 may include a display area 20 and a user interface area 22. However, in certain embodiments, such as in a touch screen, the display area 20 and the user interface area 22 may overlap. Also, in some embodiments, the display area 20 and the user interface area 22 may include a common area. In accordance with aspects of the present technique, the display area 20 of the medical imaging system 18 may be configured to display an image generated by the medical imaging system 18 based on the image data acquired via the imaging and therapy catheter 14. Additionally, the display area 20 may be configured to aid the user in defining and visualizing a user-defined therapy pathway as will be described in greater detail hereinafter. It should be noted that the display area 20 may include a three-dimensional display area. In one embodiment, the three-dimensional display may be configured to aid in identifying and visualizing three-dimensional shapes.

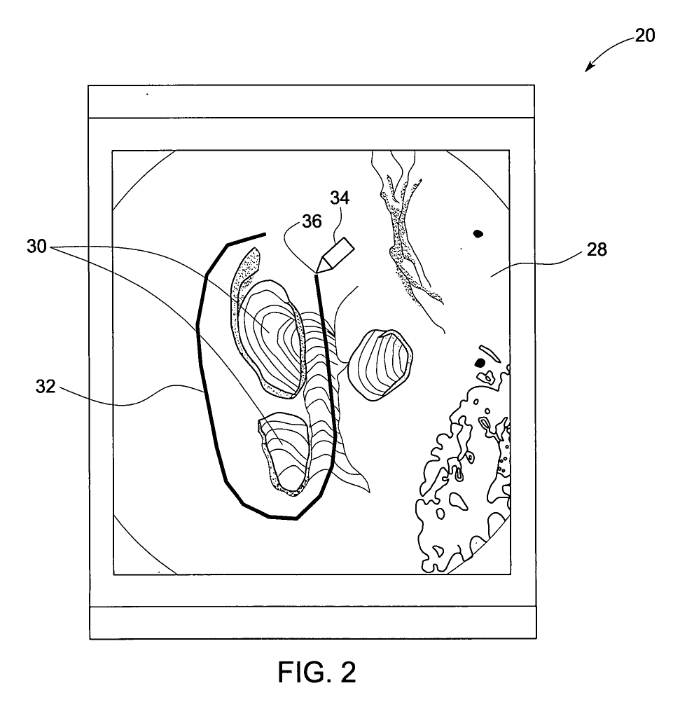

Further, the user interface area 22 of the medical imaging system 18 may include a human interface device (not shown) configured to facilitate the user in identifying the one or more regions of interest for delivering therapy using the image of the anatomical region displayed on the display area 20. The human interface device may include a mouse-type device, a trackball, a joystick, a stylus, or a touch screen configured to facilitate the user to identify the one or more regions of interest requiring therapy and define a suitable therapy pathway on the image being displayed on the display area 20. For example, the human interface device responds to a user-defined pathway by displaying a line, for instance, and will be described in greater detail with reference to FIG. 2. Additionally, the human interface device may be configured to facilitate delivery of therapy to the identified one or more regions of interest. However, as will be appreciated, other human interface devices, such as, but not limited to, a touch screen, may also be employed.

It may be noted that although the exemplary embodiments illustrated hereinafter are described in the context of an ultrasound system, other medical imaging systems such as, but not limited to, optical imaging systems, or electro-anatomical imaging systems are also contemplated for defining a therapy pathway to facilitate delivering therapy to the one or more regions of interest.

As depicted in FIG. 1, the system 10 may include an optional catheter positioning system 24 configured to reposition the imaging and therapy catheter 14 within the patient 12 in response to input from the user and relative to the defined therapy pathway. The catheter positioning system 24 will be described in greater detail hereinafter. Moreover, the system 10 may also include an optional feedback system 26 that is in operative association with the catheter positioning system 24 and the medical imaging system 18. The feedback system 26 may be configured to facilitate communication between the catheter positioning system 24 and the medical imaging system 18, as will be discussed in greater detail hereinafter.

Turning now to FIG. 2, a front view of the display area 20 of the medical imaging system 18 of FIG. 1 is illustrated. Reference numeral 28 is representative of an image generated by the medical imaging system 18 (see FIG. 1) based on the image data acquired via the imaging and therapy catheter 14 (see FIG. 1) from an anatomical region of the patient 12 (see FIG.1). Further, reference numeral 30 embodies one or more regions of interest requiring therapy identified by the user employing the displayed image 28. The user may define a therapy pathway 32 on the image 28 to select the one or more regions of interest requiring therapy. As previously noted, the user may define the therapy pathway 32 on the image 28 via a human interface device 34 such as a stylus, a trackball, a mouse, a touch screen, or a joystick, for example. In the illustrated embodiment, the human interface device is shown as including a stylus 34. It should be noted that a currently selected region of interest 36 is depicted by the current position of the stylus 34.

FIG. 3 is an illustration of an exemplary embodiment 38 of an imaging and therapy catheter 40 for use in the system 10 illustrated in FIG. 1. Further, in FIG. 3, the imaging and therapy catheter 40 is illustrated as having an imaging and therapy transducer 42. As previously noted, the imaging and therapy catheter 40 may include an imaging and therapy transducer having integrated or separate imaging and therapy components. The embodiment of the imaging and therapy catheter 40 illustrated in FIG. 3 is shown as having an integrated imaging and therapy transducer 42 having integrated imaging and therapy components. In one embodiment, the illustrated integrated imaging and therapy catheter 40 may be configured to facilitate real-time three-dimensional imaging of an anatomical region as well as deliver therapy to one or more regions in the anatomical region. For example, in the case of an integrated ultrasound imaging and therapy catheter, a real-time, three-dimensional ultrasound image may be obtained using a two-dimensional array or mechanically scanning one-dimensional array of the imaging component of the imaging and therapy transducer 42. Additionally, the integrated ultrasound imaging and therapy catheter 40 may also be configured to deliver therapy in the form of ultrasound ablation energy via a therapy component of the imaging and therapy transducer 42.

Further, reference numeral 44 is representative of a real-time three-dimensional imaged volume (RT3D). In the illustrated embodiment, the real-time three-dimensional imaged volume 44 is shown as having a pyramidal volume. In a presently contemplated configuration, reference numeral 46 is representative of a steerable beam capable of providing therapy to the identified one or more regions of interest (not shown). It should be noted that the ablation beam 46 may be steered manually or electronically. The ablation beam 46 may be steered within the three-dimensional imaged volume 44. Alternatively, the ablation beam 46 may include an ablation beam positioned in a fixed location with respect to the imaging and therapy catheter 40. The imaging and therapy catheter 40 illustrated in FIG. 3 may also include electrodes 48. The electrodes 48 may be configured to capture cardiac electrical waveforms to monitor electrical activity of the heart, for example. Additionally, in certain embodiments, the imaging and therapy catheter 40 may include a position sensor 50 disposed on a tip of the imaging and therapy catheter 40. The position sensor 50 may be configured to track motion of the imaging and therapy catheter 40 within the anatomy of the patient. Subsequently, the medical imaging system 18 (see FIG. 1) may be configured to acquire location information from the position sensor 50.

Referring now to FIG. 4, an exemplary embodiment 52 of an imaging and therapy catheter 54 having a large field of view is illustrated. The large field of view may encompass 360 degrees, in one embodiment. As depicted in FIG. 4, the imaging and therapy catheter 54 is illustrated as having an imaging and therapy transducer 56. In certain embodiments, the imaging and therapy catheter 54 may include a single imaging and therapy transducer having a large field of view. Alternatively, in other embodiments, a plurality of imaging and therapy transducers may be used in the imaging and therapy catheter 54. Further, reference numeral 58 is representative of a real-time three-dimensional imaged volume. In the illustrated embodiment, the real-time three-dimensional imaged volume 58 is shown as having a cylindrical volume. In a presently contemplated configuration, reference numeral 60 is representative of a steerable beam capable of providing therapy to the identified one or more regions of interest (not shown). The ablation beam 60 may be steered within the three-dimensional imaged volume 58. Also, as previously noted, the ablation beam 60 may be steered manually or electronically. Alternatively, the ablation beam 60 may include an ablation beam positioned in a fixed location with respect to the imaging and therapy catheter 54.

Although the embodiments illustrated in FIGS. 3 and 4 are described in the context of ultrasound ablation, it should be noted that other methods of ablation may also be employed. For instance, RF ablation may be used. Accordingly, the user may identify locations of the one or more regions of interest requiring therapy on the displayed image 28 (see FIG. 2). The medical imaging system 18 (see FIG. 1) may then be configured to control the positioning system 24 to guide the imaging and therapy catheter to the desired locations and deliver ablation energy.

FIG. 5 is a flow chart of exemplary logic 62 for imaging and providing therapy to one or more regions of interest. In accordance with exemplary aspects of the present technique, a method for imaging and providing therapy to the one or more regions of interest is presented. The method starts at step 64 where an image based on image data acquired by the medical imaging system 18 (see FIG. 1) is generated. As previously noted, the image data representative of an anatomical region of the patient 12 (see FIG. 1) may be acquired via an imaging and therapy catheter, such as imaging and therapy catheters 40 and 54 illustrated in FIG. 3 and FIG. 4 respectively. The image data may be acquired in real-time employing the imaging and therapy catheter. This acquisition of image data via the imaging and therapy catheter aids a user in assessing need for therapy in the anatomical region being imaged. In addition, mechanical means, electronic means or combinations thereof may be employed to facilitate the acquisition of image data via the imaging and therapy catheter. Alternatively, previously stored image data representative of the anatomical region may be acquired by the medical imaging system 18. The imaging and therapy catheter may include an imaging and therapy transducer. Further, an imaging orientation of the imaging and therapy catheter may include a forward viewing catheter, a side viewing catheter or combinations thereof, as previously described.

Also, the generated image, such as image 28 (see FIG. 2) is displayed on the display area 20 (see FIG. 1) of the medical imaging system 18 at step 64. In certain embodiments, the displayed image may include a real-time three-dimensional imaged volume.

Subsequently, at step 66, one or more regions of interest requiring therapy may be identified on the displayed image. In certain embodiments, the user may visually identify the one or more regions of interest using the displayed image. Alternatively, in accordance with aspects of the present technique, tissue elasticity imaging techniques may be employed to aid the user in assessing the need for therapy in the one or more regions of interest. The tissue elasticity imaging techniques may include acoustic radiation force impulse (AFRI) imaging or vibroacoustography, for example. The imaging and therapy transducer may be used to facilitate elasticity imaging. However, a separate dedicated array that is integrated onto the imaging and therapy catheter may be utilized to achieve elasticity imaging.

Following step 66, the user may define a therapy pathway, such as the therapy pathway 32 (see FIG. 2) on the displayed image at step 68. The therapy pathway is defined in response to the identified one or more regions of interest. Accordingly, in one embodiment, the therapy pathway may extend beyond a region that is capable of being imaged and treated from a single catheter position, thus requiring multiple catheter positions. Image data representative of a larger field of view may be acquired and stored. This process of acquiring and storing of image data embodying the larger field of view will be described in greater detail hereinafter. As previously noted, the user may utilize a mouse-type input device located on the user interface area 22 (see FIG. 1) of the medical imaging system 18 to draw the therapy pathway. Alternatively, the user may use a stylus, a joystick, a trackball device or a touch screen to draw the therapy pathway. The medical imaging system 18 then records the therapy pathway and displays the therapy pathway on the displayed image by overlaying the defined therapy pathway on the displayed image. The overlaying of the therapy pathway on the displayed image allows the user to visualize the therapy pathway in real-time.

It should be noted that although the embodiments illustrated are described in the context of a user-defined therapy pathway, where the user manually delineates the therapy pathway, an automatically defined therapy pathway is also contemplated. The imaging and therapy system 10 (see FIG.1) may be configured to provide a system generated proposed therapy pathway based on selected characteristics of the image data. Accordingly, the system 10 may be configured to automatically identify one or more regions in the imaged volume requiring therapy based on the selected characteristics. Subsequently, the system 10 may also automatically propose a therapy pathway based on locations of the identified one or more regions requiring therapy. The selected characteristics may include mechanical properties of tissues, such as, but not limited to, a density, brightness, tissue stiffness or combinations thereof which may be indicative or representative of certain diseases that would respond to therapy.

Step 70 depicts a process of delivering therapy to the identified one or more regions of interest in accordance with the defined pathway. During step 70, the medical imaging system 18 processes the therapy pathway defined at step 68 and converts the defined therapy pathway into a series of actions resulting in execution of the therapy in accordance with the therapy pathway defined in step 68. The series of actions resulting in execution of the therapy depend on the specific embodiment and will be described in greater detail hereinafter. Accordingly, the medical imaging system 18 is configured to determine location information of each of the one or more regions of interest. The medical imaging system 18 may be configured to determine location information of each of the one or more regions of interest by processing the defined therapy pathway in combination with known location information of each point on the displayed image relative to the known positions of the imaging and therapy components of the catheter.

With continuing reference to step 70, if the one or more regions of interest are located within the field of view of the imaging and therapy transducer, the medical imaging system 18 may be configured to deliver therapy through the therapy component of the imaging and therapy transducer in the imaging and therapy catheter to the identified one or more regions of interest. In one embodiment, the therapy may include high intensity focused ultrasound (HIFU) energy. The medical imaging system may deliver the therapy by steering an ablation beam, such as ablation beams 46 (see FIG. 3) and 60 (see FIG. 4) within the imaged volume. Accordingly, in one embodiment, the ablation beam may include a steerable ablation beam. The ablation beam may be steered using conventional phasing techniques that include phasing excitation of the ablation array to ensure propagation of the ultrasound beam in a desirable direction. It should be noted if the ablation beam is steerable, the one or more regions of interest within the field of view of the imaging and therapy transducer may be ablated without repositioning the imaging and therapy catheter, thereby advantageously resulting in less movement of the imaging and therapy catheter within the patient. Also, if the imaging and therapy transducer has a large field of view, such as the imaging and therapy catheter 54 illustrated in FIG. 4, the one or more regions of interest may be ablated while the imaging and therapy catheter is positioned at a single location.

Alternatively, if the ablation beam is fixed, the imaging and therapy catheter may need to be repositioned prior to delivering therapy. A check may then be carried out at an optional step to verify if the one or more regions of interest requiring therapy are positioned within a field of view of the imaging and therapy transducer. If the one or more regions of interest requiring therapy are currently positioned outside the field of view of the imaging and therapy transducer, then the imaging and therapy catheter may be repositioned to include the one or more regions of interest within the field of view of the imaging and therapy transducer. This repositioning of the imaging and therapy catheter facilitates imaging and delivering therapy to the one or more regions of interest that are currently located outside the field of view of the imaging and therapy catheter. Additionally, if the one or more regions of interest requiring therapy includes a three-dimensional shape, repositioning of the imaging and therapy catheter may be required to cover the three-dimensional shape.

Furthermore, in accordance with aspects of the present technique, three-dimensional volumes with a larger field of view may be assembled by employing an imaging and therapy catheter having a limited field of view. Moreover, information regarding the three-dimensional volumes and defined therapy pathways may be stored in memory, for example. Consequently, a composite image may be generated by assembling several images, where the images are representative of a plurality of positions of the imaging and therapy catheter. The composite image may be stored in memory. This assembly of three-dimensional volumes with a larger field of view may be achieved by tracking image characteristics, such as speckle targets, or other image features. The current field of view imaged by the imaging and therapy catheter may then be registered with the larger stored three-dimensional volume in real-time. This allows a user to identify where the localized treatment pathway is located with respect to an overall treatment pathway when the overall treatment pathway extends beyond what is visible at a single given instant. In one embodiment, one or more regions of interest selected by the user may be located outside a field of view of the current position of the imaging and therapy catheter. The imaging and therapy catheter may then be accordingly repositioned to include within the current field of view the one or more regions of interest presently located outside the field of view of the imaging and therapy catheter, while moving the treated one or more regions of interest out of the field of view.

In one embodiment, the imaging and therapy catheter may include a position sensor 50 (see FIG. 3) disposed on a tip of the imaging and therapy catheter. As previously noted, the position sensor 50 may be configured to track motion of the imaging and therapy catheter within the anatomy of the patient. Subsequently, the medical imaging system may be configured to acquire location information from the position sensor.

In certain embodiments, the imaging and therapy catheter may be repositioned manually. Alternatively, the imaging and therapy catheter may be automatically repositioned to image and deliver therapy to the one or more regions of interest employing the catheter positioning system 24 illustrated in FIG. 1. The catheter positioning system 24 may include a sub-system (not shown) that may be configured to provide location information regarding a tip of the imaging and therapy catheter. As used herein, “tip” of the imaging and therapy catheter is representative of a length of about 10 centimeters or less from a distal end of the imaging and therapy catheter. In certain embodiments, the tip of the imaging and therapy catheter also may include the imaging and therapy components of the imaging and therapy catheter. Further, the catheter positioning system 24 may also include an actuating sub-system (not shown) that may be configured to actuate the tip of the catheter. Accordingly, the location information associated with the one or more regions of interest currently located outside the field of view of the imaging and therapy catheter may be communicated to the catheter positioning system 24 via the feedback system 26 (see FIG. 1). The user may utilize the human interface device to provide information regarding location of a subsequent volume to be imaged to the catheter positioning system 24 via the feedback system 26, for example. Consequently, the catheter positioning system 24 may be configured to automatically reposition the imaging and therapy catheter to the desirable location thereby ensuring that the one or more regions of interest are positioned within the field of view of the imaging and therapy catheter.

It should also be noted that the process of delivering therapy may be preferably performed in real-time. Accordingly, the imaging and therapy catheter may deliver therapy in real-time to the one or more regions of interest in response to input from the user. In other words, therapy may be delivered to the one or more regions of interest while the user is drawing the therapy pathway on the displayed image. In view of this, the medical imaging system may be configured to track the defined therapy pathway as it is drawn on the displayed image. Subsequently, the imaging and therapy catheter may be configured to steer the ablation beam to deliver the therapy. Alternatively, the medical imaging system may be configured to deliver the therapy to the one or more regions of interest after the therapy pathway has been drawn to a predetermined extent.

Additionally, the efficacy of the therapy after it is delivered may be monitored via the use of the tissue elasticity imaging techniques. Also, the medical imaging system may be configured to use imaging processing algorithms to accurately monitor the therapy treated sites. The imaging processing algorithms may also be used to monitor motion of the tissue being imaged and treated. In certain embodiments, the image processing algorithms may include speckle tracking algorithms or other correlation-based algorithms.

It should also be noted that the procedure of imaging and providing therapy to the one or more regions of interest requiring therapy may be executed from a remote location once the imaging and therapy catheter has been positioned within the patient. The user may access the image data from a remote location, which may advantageously assist the user in remotely monitoring the delivery of therapy. The image data acquired via the imaging and therapy catheter may be transmitted via a wireless medium to a central monitoring system that may be located within a caregiving facility. The user may then access the central monitoring system to remotely view the image data, identify the one or more regions requiring therapy, and deliver the therapy accordingly. In general, displays, printers, workstations, and similar devices supplied within the system may be local to the image acquisition components, or may be remote from these components, such as elsewhere within caregiving facility, or in an entirely different location, linked to the medical imaging system via one or more configurable networks, such as the Internet, virtual private networks, and so forth.

As will be appreciated by those of ordinary skill in the art, the foregoing example, demonstrations, and process steps may be implemented by suitable code on a processor-based system, such as a general-purpose or special-purpose computer. It should also be noted that different implementations of the present technique may perform some or all of the steps described herein in different orders or substantially concurrently, that is, in parallel. Furthermore, the functions may be implemented in a variety of programming languages, such as C++or Java. Such code, as will be appreciated by those of ordinary skill in the art, may be stored or adapted for storage on one or more tangible, machine readable media, such as on memory chips, local or remote hard disks, optical disks (that is, CD's or DVD's), or other media, which may be accessed by a processor-based system to execute the stored code. Note that the tangible media may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

The various methods of imaging and providing therapy and the systems for imaging and providing therapy described hereinabove dramatically enhance efficiency of the process of delivering therapy, such as ablation, by integrating the imaging, therapy, and mapping aspects of the procedure, thereby advantageously eliminating the need for pre-case CT/MRI and static electroanatomical mapping systems. In addition, exposure to harmful ionizing radiation required with current fluoroscopic imaging methods is eliminated.

Also, the use of the human interface device greatly aids the user in identifying the one or more regions requiring therapy and defining the therapy pathway on the displayed image representative of the imaged anatomical region, rather than having to manually manipulate an RF ablation catheter to physically contact each region on the anatomy to be treated. Consequently, definition of the therapy pathway is greatly improved resulting in lower collateral damage to the tissue of the anatomy being treated. Further, the imaging and therapy transducer with the steerable ablation beam advantageously results in less movement of the imaging and therapy catheter, thereby greatly increasing patient comfort.

Further, employing the techniques of imaging and providing therapy described hereinabove facilitates building cost effective imaging and therapy systems due to reduction in the number of operators required to operate the imaging and therapy system. Current systems require multiple operators to operate each of the ablation system, fluoroscopic imaging system, and the two-dimensional ultrasound imaging catheter, while the imaging and therapy system described hereinabove is configured to image the anatomy and monitor the delivery of therapy with a single device. Furthermore, the imaging and therapy system described hereinabove may be advantageously be operated by a single operator.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A system for imaging and providing therapy to one or more regions of interest, the system comprising:
an imaging and therapy catheter configured to image an anatomical region to facilitate assessing need for therapy in one or more regions of interest within the anatomical region and delivering therapy to the one or more regions of interest within the anatomical region; and
a medical imaging system operationally coupled to the catheter and having a display area and a user interface area, wherein the medical imaging system is configured to facilitate definition of a therapy pathway to facilitate delivering therapy to the one or more regions of interest.

2. The system of claim 1, wherein the imaging and therapy catheter comprises a real-time imaging and therapy transducer.

3. The system of claim 2, wherein the imaging and therapy transducer comprises integrated imaging and therapy components.

4. The system of claim 1, wherein the therapy comprises ablation, percutaneous ethanol injection, cryotherapy, laser-induced thermotherapy, delivery of tools for gene therapy, surgical tools or combinations thereof.

5. The system of claim 1, further comprising a catheter positioning system configured to reposition the catheter automatically or in response to input from a user and relative to the defined therapy pathway.

6. The system of claim 5, wherein the catheter positioning system comprises a tip position sensor configured to provide location information of a tip of the catheter and a mechanism configured to actuate the tip of the catheter.

7. The system of claim 1, further comprising a feedback system in operative association with the catheter positioning system and the medical imaging system, wherein the feedback system is configured to facilitate communication between the catheter positioning system and the medical imaging system.

8. The system of claim 1, wherein the medical imaging system comprises an ultrasound system, an optical imaging system, an electro-anatomical imaging system or combinations thereof.

9. The system of claim 1, wherein the user interface area of the medical imaging system comprises a human interface device configured to facilitate the user to identify the one or more regions of interest for directing therapy using an image of the anatomical region displayed on the display area of the medical imaging system.

10. The system of claim 1, wherein the display area includes a three-dimensional display area configured to aid in identifying one or more regions of interest and in visualizing three-dimensional shapes.

11. The system of claim 1, wherein the imaging and therapy catheter comprises a forward viewing catheter, a side viewing catheter or combinations thereof.

12. The system of claim 1, wherein the medical imaging system is configured to provide control signals to the imaging and therapy catheter to excite the therapy component of the imaging and therapy transducer and deliver therapy to the one or more regions of interest.

13. The system of claim 1, further configured to provide a system generated proposed therapy pathway based on selected characteristics of the image data.

14. The system of claim 13, wherein the selected characteristics comprise a brightness, a density, a tissue stiffness, or combinations thereof.

15. A method for imaging and providing therapy to one or more regions of interest, the method comprising:
generating an image from acquired image data for display on a display area of a medical imaging system;
identifying one or more regions of interest requiring therapy on the displayed image;
defining a therapy pathway in response to the identified one or more regions of interest; and
delivering therapy to the one or more regions of interest in accordance with the defined therapy pathway.

16. The method of claim 15, further comprising acquiring the image data via an imaging and therapy catheter to facilitate assessing need for therapy.

17. The method of claim 16, wherein the imaging and therapy catheter comprises an imaging and therapy transducer.

18. The method of claim 15, wherein the defining step comprises drawing the therapy pathway on the displayed image via a human interface device.

19. The method of claim 18, further comprising determining location information of the one or more regions of interest.

20. The method of claim 19, further comprising communicating the location information via a feedback system between a catheter positioning system and the medical imaging system.

21. The method of claim 20, further comprising repositioning the imaging and therapy catheter to a desirable location to facilitate inclusion of the one or more regions of interest within a field of view of the imaging and therapy transducer.

22. The method of claim 15, further comprising providing a system generated proposed therapy pathway based on selected characteristics of the image data.

23. A computer readable medium comprising one or more tangible media, wherein the one or more tangible media comprise:
code adapted to generate an image from acquired image data for display on a display area of a medical imaging system;
code adapted to identify one or more regions of interest requiring therapy on the displayed image;
code adapted to define a therapy pathway in response to the identified one or more regions of interest; and
code adapted to deliver therapy to the one or more regions of interest in accordance with the defined therapy pathway.

24. The computer readable medium, as recited in claim 23, further comprising code adapted to acquire the image data via an imaging and therapy catheter to facilitate assessing need for therapy.

25. The computer readable medium, as recited in claim 23, further comprising code adapted to determine location information of the one or more regions of interest and communicate the location information via a feedback system between a catheter positioning system and the medical imaging system.

26. The computer readable medium, as recited in claim 25, further comprising code adapted to reposition the imaging and therapy catheter to a desirable location to facilitate inclusion of the one or more regions of interest within a field of view of an imaging and therapy transducer, wherein the imaging and therapy catheter comprises the imaging and therapy transducer.

27. A system for imaging and providing therapy to one or more regions of interest, the system comprising:
an imaging and therapy catheter configured to image an anatomical region to facilitate assessing the need for therapy in one or more regions of interest within the anatomical region and delivering therapy to the one or more regions of interest within the anatomical region;
a medical imaging system operationally coupled to the catheter and having a display area and a user interface area, wherein the medical imaging system is configured to facilitate defining a therapy pathway to facilitate delivering therapy to the one or more regions of interest;
an image generation sub-system for receiving acquired image data, generating an image of the anatomical region and displaying the image on the display area of the medical imaging system; and
an operator console for identifying the one or more regions of interest on the displayed image.

28. The system of claim 27, further comprising a catheter positioning system in operative association with the imaging and therapy catheter and configured to reposition the catheter automatically or in response to input from a user and relative to the defined therapy pathway.

29. The system of claim 27, further comprising a feedback system operationally coupled to the catheter positioning system and the medical imaging system, wherein the feedback system is configured to facilitate communication between the catheter positioning system and the medical imaging system.

30. The system of claim 27, wherein the medical imaging system is configured to provide control signals to the imaging and therapy catheter to excite a therapy component of the imaging and therapy transducer and steer an ablation beam to deliver therapy to the one or more regions of interest, wherein the imaging and therapy catheter comprises the imaging and therapy transducer.

31. The system of claim 27, further configured to provide a system generated proposed therapy pathway based on selected characteristics of the image data.

32. The system of claim 27, wherein the system is configured to generate a composite image by assembling images from a plurality of imaging and therapy catheter positions and store the composite image.

33. The system of claim 32, wherein one or more regions of interest are located outside a field of view of a current position of the imaging and therapy catheter.

34. The system of claim 33, wherein the system is configured to reposition the imaging and therapy catheter to follow the therapy pathway.
